(19) United States
(12) Patent Application Publication
(10) Pub. No.: US 2008/0200837 A1
(43) Pub. Date: Aug. 21, 2008

(54) DISPOSABLE, CLOSED BLOOD SAMPLING SYSTEM FOR USE IN MEDICAL CONDUIT LINE

(76) Inventors: John A. Frazier, Costa Mesa, CA (US); Greg Bak-Boychuk, Laguna Niguel, CA (US); Mark Wright, Carlsbad, CA (US); Morgan T. McKeown, Irvine, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/675,578

(22) Filed: Feb. 15, 2007

(51) Int. Cl. A61B 5/155

(52) U.S. Cl. 600/573

(57) ABSTRACT

A disposable, closed fluid sampling system for use with a medical conduit line, especially for taking blood samples from a pressure monitoring line. The conduit line has at least one fluid sampling site interposed between a distal segment of tubing and a proximal segment of tubing. The system includes a disposable subsystem having a bypass cannula adapted to engage the sampling site, a reservoir for drawing a prime volume of fluid past the sampling site, and a fluid sampling container. A 3-way stopcock may connect the reservoir, bypass cannula, and sampling vessel for controlling the sampling procedure. The bypass cannula may simultaneously place the subsystem into fluid communication with an inner chamber of the sampling site and exclude the proximal segment of the conduit line. Alternatively, a stopcock immediately adjacent the sampling site in the proximal segment may be utilized. A method of using the system includes attaching the disposable subsystem, opening the reservoir to the distal segment while excluding the proximal segment, drawing a prime volume of fluid into the reservoir, opening the sampling vessel to the distal segment and drawing a sample, re-infusing the prime volume into the distal segment, and removing and discarding the subsystem. The reservoir remains attached between the prime volume draw and re-infusion and is thus “closed.”

### FIELD OF THE INVENTION

The present invention relates to blood sampling systems and, in particular, to a disposable blood sampling system in a medical conduit line such as a pressure monitoring line.

### BACKGROUND OF THE INVENTION

In a hospital setting there is always the need to monitor patient health through the evaluation of blood chemistry profile. The simplest method employed in the hospital is to use a syringe carrying a sharpened cannula at one end and insert that cannula into a vein or artery to extract a blood sample from the patient. Blood is drawn using a syringe or more easily into an evacuated vessel. Patients that are in the critical care units or the operating room sometimes require as many as twelve blood draws a day which can be quite uncomfortable. Moreover, such frequent sampling injections carry the risk to the clinician of needle sticks and blood exposure, and potentially subject the patient to airborne bacteria and viruses which can enter the bloodstream through the opening made by the sharpened cannula.

One way to obtain a blood sample is to draw the blood from a catheter that is already inserted in the patient, either in a central venous line, such as one placed in the right atrium, or in an arterial line. Typically, existing injection sites for arterial or venous drug infusion or pressure monitoring lines are used to take periodic blood samples from the patient. Conventional mechanisms for drawing blood from the lines used for infusion or pressure monitoring utilize a plurality of stopcock mechanisms that block flow from the infusion fluid supply or from the pressure column drip supply, while allowing blood to flow from the patient into a collecting syringe connected to a port formed in one of the stopcocks. Typically, a blunt cannula through a slit septum is used to remove the danger of sticking the nurse or clinician, in a so-called “needle-less” system.

Most early systems required a two-step operation where a first sample of fluid, typically between 2-12 ml in volume for intensive care environments was withdrawn into the sampling syringe and discarded. This first sample potentially included some of the infusion fluid and thus would be an unreliable blood chemistry measurement sample. After the initial sample had been discharged, the second sample was pure blood from the artery or vein and was typically re-infused to the patient.

Of course, the possibility of discarding blood along with saline is a drawback, especially in anemic patients, and so-called closed systems that remain connected to the conduit line and preserve the initial fluid draw were developed. Examples of these can be seen in U.S. Pat. No. 4,673,386 to Gordon, and more recently in U.S. Pat. No. 5,961,472 to Swendson. Commercial closed systems such as the Edwards VAMP® and VAMP Plus® Venous Arterial blood Management Protection systems of Edwards Lifesciences in Irvine, Calif. feature a dedicated syringe-like reservoir incorporated in the tubing line from the patient that can draw fluid past a sampling port. The line continues past the reservoir to a proximal source of flushing fluid and a pressure transducer. (The standard directional nomenclature is that proximal is toward the clinician, or away from the patient, and distal is toward the patient). The clearing volume is held in the in-line reservoir, and not set aside in a syringe for discard or re-infusion later.

When a blood sample is to be taken, the flow of flushing or infusion fluid is halted by turning the handle of a reservoir stopcock valve. The nurse or clinician then withdraws an amount of fluid into the reservoir chamber and distal line sufficient to pull pure blood past one or more sampling sites and closes the reservoir stopcock. Desirably, the flow line includes a sampling site near the patient more often used in the ICU, and/or one farther away, close to the reservoir, and used in the OR when the space immediately around the patient is at a premium. To avoid needle sticks, blunt cannulas are used to draw blood. Also, the sampling sites are desirably designed to ensure a complete flush after the sample is taken. The stopcock valve is then opened so that the volume within the reservoir can be re-infused back into the patient, and the flushing drip and pressure monitoring resumes.

In reservoir systems the nurse or technician must manipulate the reservoir, then let go of it to take the blood sample, and then grasp it again to re-infuse the prime volume, all of which is relatively inconvenient. Furthermore the continuing presence of the reservoir dangling from the pressure monitoring line is undesirable as it is only infrequently used and can become tangled with bedding or with other equipment. Finally, because the reservoir remains in place and is used multiple times, it must include a contamination shield to isolate the reservoir plunger, and such a device is costly compared to a simple syringe.

A minimum quality of system frequency response in the blood sampling/pressure monitoring systems described above is necessary for reliable blood pressure measurements. The pressure transducer typically includes a diaphragm exposed to the in-line fluid on one side and has a device for measuring deflection of the diaphragm on the other. Adding components to the flow line, such as sampling sites with elastomeric septums or a dedicated reservoir with a rubber-tipped plunger, and/or increasing the length of tubing contributes to signal degradation. Often only one sampling site is provided for the ICU or OR to avoid unduly degrading the signal response.

In view of the foregoing, there is a need for a blood sampling system, especially used in conjunction with a pressure transducer, that is more convenient and safer to use.

### SUMMARY OF THE INVENTION

In one aspect, the present invention provides a medical system for sampling of a fluid system of a patient for use with a conduit line. The conduit line includes a proximal segment adapted to be supplied with a physiological fluid and a distal segment adapted to be in communication with the fluid system of a patient. A fluid sampling site in the conduit line defines a housing having a distal port connected to the distal segment and a proximal port connected to the proximal segment. Fluid may flow freely through a chamber in the housing between the proximal and distal ports, and the sampling site further defines a sampling port open to the chamber and closed from the exterior by an elastomeric seal. The medical system comprises a flowthrough bypass cannula having a body shaped to engage the sampling port. The cannula also has a bypass probe defining a throughbore and sized to pierce the elastomeric seal, project into the chamber and place the throughbore into fluid communication with the distal port of the sampling site while occluding the proximal port from the throughbore. Consequently, a fluid sample may be drawn into the bypass cannula from the distal segment of the conduit line to the exclusion of the proximal segment. In a preferred embodiment, the bypass cannula and sampling site include mating structure such that they engage with a positive snap fit.

In one embodiment, the bypass probe terminates in a tip that contacts a passageway opening in the fluid sampling site housing leading to one of the distal or proximal ports when the bypass cannula engages the sampling site, and the tip is tapered and matches the passageway opening. In another embodiment, the bypass probe terminates in a closed tip that contacts and occludes a passageway opening in the fluid sampling site housing leading to the proximal port when the bypass cannula engages the sampling site, and the bypass probe includes at least one side opening open to the chamber in the sampling site housing. The closed bypass probe tip may be at least partly compressible to enhance the seal between the tip and the passageway opening. Alternatively, the bypass probe defines a continuous tubular member and terminates in an open tip that contacts a passageway opening in the fluid sampling site housing leading to the distal port when the bypass cannula engages the sampling site. Furthermore, a sealing member may be positioned in the passageway opening that contacts and seals against the open tip of the bypass probe to enhance the seal therebetween.

Another aspect of the present invention is a system for sampling fluid from a medical conduit line including a disposable subsystem. The system includes a conduit line with a proximal segment adapted to be supplied with a physiological fluid and a distal segment adapted to be in communication with a fluid system of a patient. A fluid sampling site in the conduit line defines an internal chamber in communication with all of a proximal port connected to the proximal segment, a distal port connected to the distal segment, and a sampling port closed by an elastomeric seal. Further has a disposable sampling subsystem for engaging the fluid sampling site. The sampling subsystem includes a bypass cannula adapted to engage the sampling port of the sampling site and simultaneously occlude the proximal port therein. The sampling subsystem also includes a stopcock having three ports, a first port in communication with the bypass cannula, a fluid reservoir connected to a second port of the stopcock, and a fluid sampling vessel in fluid communication with the third port of the stopcock. Manipulating the stopcock into a first position enables fluid communication between the first and second ports so that fluid may be drawn into the reservoir from the distal segment. Manipulating the stopcock into a second position enables fluid communication between the first and third ports so that fluid may be drawn into the fluid sampling vessel from the distal segment. The system further may include a pressure transducer connected to the proximal segment of the conduit line for sensing the pressure of the fluid system of a patient through the fluid in the conduit line. An optional component of the system is a bubble trap connected to the bypass cannula between the sampling site and reservoir that prevents bubbles from entering the sampling site from the bypass cannula.

In one embodiment, the bypass cannula includes a bypass probe terminating in a closed tip that contacts and occludes a passageway opening in the fluid sampling site housing leading to the proximal port when the bypass cannula engages the sampling site, and the bypass probe includes at least one side opening open to the chamber in the sampling site housing. In an alternative embodiment, the bypass cannula includes a continuous tubular bypass probe terminating in an open tip that contacts a passageway opening in the fluid sampling site housing leading to the distal port when the bypass cannula engages the sampling site. A still further alternative includes a stopcock positioned adjacent the proximal port of the sampling site for occluding the proximal port when fluid is drawn into and expelled from the reservoir and drawn into the fluid sampling container.

The present invention also provides a method for sampling blood from a medical fluid conduit line using a disposable subsystem, the conduit line having a proximal segment supplied with a physiological fluid and a distal segment in communication with the blood system of a patient. The conduit line also has a fluid sampling site defining an internal chamber in communication with all of: a proximal port connected to the proximal segment, a distal port connected to the distal segment, and a sampling port closed by an elastomeric seal. The method includes the steps of:
providing a disposable sampling subsystem for engaging the fluid sampling site including a flowthrough bypass cannula having a probe, a fluid reservoir, and a fluid sampling container;
engaging the bypass cannula with the sampling site so that the probe passes through the elastomeric seal;
opening fluid communication between the bypass cannula and reservoir while closing fluid communication between the bypass cannula and fluid sampling container;
closing fluid communication between the bypass cannula and proximal segment of the conduit line and drawing fluid into the reservoir from the distal segment through the bypass cannula until blood enters the sampling site;
opening fluid communication between the bypass cannula and fluid sampling vessel while closing fluid communication between the bypass cannula and reservoir;
drawing blood into the fluid sampling vessel from the distal segment;
maintaining the bypass cannula engaged with the sampling site until a sample of blood is drawn into the fluid sampling container; and
detaching the bypass cannula from the sampling site and disposing of the sampling subsystem.

The method desirably also includes providing a pressure transducer connected to the proximal segment of the conduit line and sensing the pressure of the fluid system of a patient through the fluid in the conduit line.

In one embodiment, the disposable sampling subsystem includes a stopcock having three ports, a first port in communication with the bypass cannula, a second ports in communication with the fluid reservoir, and a third port in communication with the fluid sampling container, wherein following steps of the method are enabled by manipulating the stopcock:
opening fluid communication between the bypass cannula and reservoir while closing fluid communication between the bypass cannula and fluid sampling container; and
opening fluid communication between the bypass cannula and fluid sampling vessel while closing fluid communication between the bypass cannula and reservoir.

In one version of the method the step of opening fluid communication between the bypass cannula and reservoir while closing fluid communication between the bypass cannula and fluid sampling vessel is accomplished by the step of engaging the bypass cannula with the sampling site. The probe of the bypass cannula may have a size sufficient to contact a passageway opening leading to the proximal port of the sampling site, and exclude fluid communication between the proximal port and the distal port by, for example, occluding the passageway opening.

The method may alternatively include providing a stopcock adjacent the proximal port of the sampling site for occluding the proximal port when fluid is drawn into and expelled from the reservoir and drawn into the fluid sampling container.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

Features and advantages of the present invention will become appreciated as the same become better understood with reference to the specification, claims, and appended drawings wherein:

FIGS. 1A-1C are schematic diagrams of blood sampling systems within dedicated closed reservoirs within pressure monitoring lines of the prior art;

FIGS. 2A-2B are schematic diagrams of two different configurations of a blood sampling system of one embodiment of the present invention within a pressure monitoring line;

FIG. 3 illustrates a typical hospital room setup of a pressure monitoring system for a patient incorporating two fluid sampling ports of one embodiment of the present invention;

FIG. 4 schematically illustrate a fluid sampling system in conjunction with the fluid sampling ports of FIG. 3;

FIG. 5 is an exploded sectional view of the main fluid sampling components of one embodiment of the present invention;

FIG. 6 is a partial sectional view of a flowthrough bypass cannula of the fluid sampling system of one embodiment of the present invention engaged with a fluid sampling site and illustrating the occlusion of a proximal segment of the pressure monitoring line;

FIGS. 7A-7E are schematic views illustrating a sequence of steps involved in taking a fluid sample in accordance with one embodiment of the present invention;

FIGS. 8A-8C are perspective assembled and exploded views of an exemplary bypass cannula and a sampling site combination of one embodiment of the present invention;

FIG. 9A is a top plan view of the bypass cannula and sampling site combination of FIGS. 8A-8C;

FIGS. 9B and 9C are vertical sectional views along orthogonal axes through the centerline of the bypass cannula and sampling site combination, taken along respective section, lines shown in FIG. 9A;

FIG. 10 is a vertical sectional view through an alternative bypass cannula and sampling site combination of one embodiment of the present invention;

FIG. 11 is a vertical sectional view through another alternative bypass cannula and sampling site combination of one embodiment of the present invention;

FIGS. 12A and 12B illustrates a still further alternative bypass cannula and sampling site combination, as well as an exemplary bubble trap that may be used therewith; and

FIG. 13 is an alternative disposable fluid sampling system of one embodiment of the present invention that permits fluid to be drawn into and expelled from a removable reservoir to and from a distal segment of a conduit line while occluding a proximal segment.

### DETAILED DESCRIPTION OF THE PREFERRED EMBODIMENTS

The present invention provides an improved blood sampling system, desirably for use in a pressure monitoring line. As mentioned above, continuous or periodic blood pressure monitoring is a common and extremely useful tool in the intensive care or operating room. However, it should be mentioned that the apparatuses and methods described herein could be utilized in conjunction with any fluid system of a patient which would benefit from pressure monitoring. For instance, intracranial pressures could be monitored and cerebrospinal fluid samples taken by placing the system described herein in fluid communication with an intraventricular catheter. Therefore, the appended claims cover the sampling and monitoring of any fluid system within a patient unless otherwise specified. Additionally, certain aspects of the present invention may be useful for taking fluid samples in conduit lines connected to the patient that do not include pressure monitoring, such as from existing injection lines for arterial or venous drug infusion.

The present invention comprises an improved sampling system especially useful for sampling blood in the operating room (OR), intensive care unit (ICU) or critical care unit (CCU). The system is closed as some in prior systems, for example the VAMP Plus® Venous Arterial blood Management Protection system available from Edwards Lifesciences of Irvine, Calif., because the prime volume drawn in to a reservoir may be returned to the patient. However, one embodiment of the present invention also provides an option of discarding the prime volume, and does not include a “dedicated” in-line reservoir.

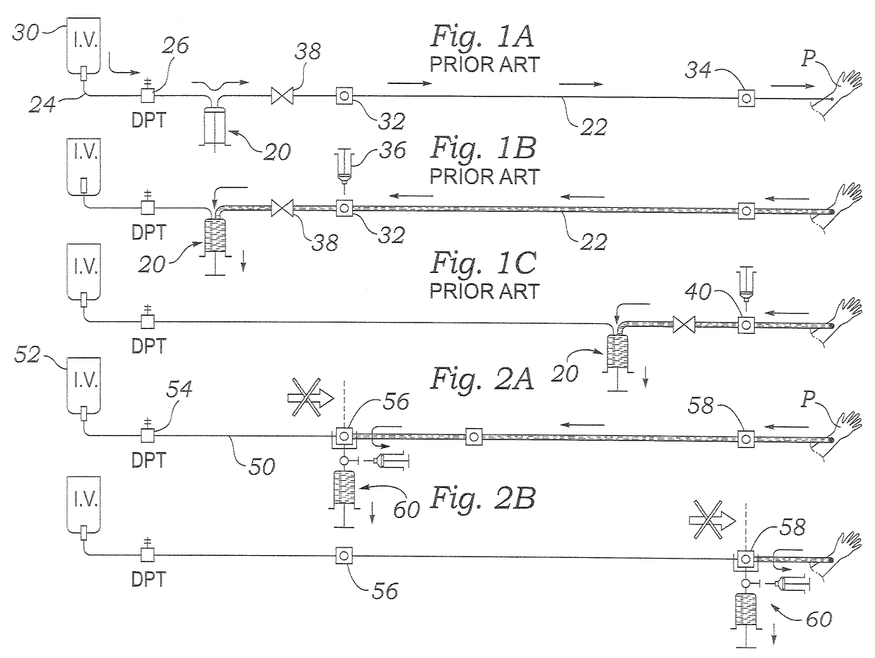

FIGS. 1A-1C are schematic diagrams of closed blood sampling systems within pressure monitoring lines of the prior art. As explained above, a “closed” system typically features a dedicated in-line reservoir (i.e., one that remains connected), therefore avoiding “breaking” (or opening) the conduit line to pull the pure blood across the sampling site. This eliminates any potential contamination of the blood or contamination of the environment from the blood, such as if a removable syringe was used and set aside for later re-infusion. The systems illustrated in FIGS. 1A-1C all include a dedicated reservoir 20 mounted within a fluid conduit line or tubing connected to a fluid system of the patient P, much like the VAMP Plus® Venous Arterial blood Management Protection system available from Edwards Lifesciences.

FIG. 1A illustrates a pressure monitoring system where the placement of the reservoir 20 defines a distal segment 22 and a proximal segment 24 of the conduit line. The conduit line is primarily medical grade pressure tubing. The system includes a disposable pressure transducer (DPT) 26 in the proximal segment 24 which measures pressure pulses within the fluid column in the conduit line derived from a patient to which the distal segment 22 connects. Although not shown, the distal end of the distal segment 22 typically includes a luer connector which engages a mating luer connector on an in-dwelling catheter or cannula placed in a vein or artery of the patient. In this manner, the pulse and blood pressure of the patient transmits through the fluid column within the conduit line to the pressure transducer 26.

Pressure lines such as shown in FIG. 1A typically make use of relatively stiff tubing primed with a suitable physiological fluid such as saline or 5% dextrose solution from a supply bag 30 (denoted “I.V.”) as a pressure column. For adults, a bag pressurized with air surrounds the fluid supply bag to maintain a constant pressure differential in the line urging fluid toward the patient through a restrictor orifice. The slow drip of physiological fluid, as indicated by the flow arrows in FIG. 1A, flushes the line to prevent clotting. Some transducers such as the TruWave® Disposable Pressure Transducer from Edwards Lifesciences include a flush device that also can be used for sending transient pressure waves through the line. A Snap-Tab™ device of the TruWave® is a rubber tab which when pulled and then released sends a square wave through the pressure column to check the inherent frequency response of the entire system, which includes the tubing and any components attached thereto.

When the pressure line is also used for fluid sampling it features at least one and preferably two sampling sites 32, 34 in the proximal segment 24. A proximal sampling site 32 is located near the reservoir 20, often 5 or 6 feet (152-178 cm) from the patient. The distal sampling site 34 is located close to the distal end of the conduit line. The operators (clinicians) who draw blood samples can be generally categorized in two groups: 1) OR nurses and anesthesiologists, and 2) CCU or ICU nurses. Each grouping has different requirements regarding position of the sampling sites. In the OR, access near the patient is limited; therefore the preferred sampling site location is near the IV pole, so the proximal sampling site 32 is preferred. On the other hand, in the CCU or ICU nurses prefer to take blood as close to the patient as possible, so the distal sampling site 34 is preferred.

FIG. 1B shows one step in operation of the sampling system wherein a prime volume of fluid has been drawn into the reservoir 20 from the distal segment 22 of the conduit line. The reservoir 20 has sufficient capacity to draw all of the saline and mixed saline and blood into its chamber such that pure blood extends from the patient past one or both of the sampling sites 32, 34. In the illustrated embodiment, enough prime volume has been drawn into the reservoir 20 such that a sample can be taken at the proximal sampling site 32, as indicated by the schematic sampling syringe 36, though of course a sample could be taken at the distal site 34. Prior to taking the sample, a stopcock 38 between the reservoir 20 and sampling site 32 is closed to prevent drawing any fluid into the sampling syringe 36 from the reservoir 20, thus insuring an undiluted sample. After taking a sample, the nurse withdraws the syringe 36 from the sample site 32, opens the stopcock 38, and depresses the plunger of the reservoir 20 so that the entire prime volume infuses back to the patient.

FIG. 1C illustrates an alternative pressure monitoring/fluid sampling conduit line wherein a dedicated reservoir 20 is located close to the patient and there is only one sampling site 40. This system functions in a similar manner to the one described above, but is typically only used in the CCU or ICU because of the proximity of the sampling site 40 to the patient, which would get in the way in the OR.

FIGS. 2A and 2B are schematic views of a pressure monitoring/fluid sampling system of one embodiment of the present invention. As in the prior art systems, a conduit line 50 extends between an injection site in a vein or artery of the patient to a source of physiological fluid 52. A pressure transducer 54 connects to the fluid column within the conduit line 50 near the proximal end, and the system includes a proximal sampling site 56 and a distal sampling site 58, desirably at the same locations as those shown in FIG. 1A.

The system of FIGS. 2A and 2B does not include a dedicated in-line reservoir or stopcock as described above. Instead, a removable fluid sampling subsystem 60 is designed to engage either of the sampling sites 56, 58, but does not remain attached to the conduit line when not in use. FIG. 2A illustrates engagement of the subsystem 60 with the proximal sampling site 56, while FIG. 2B shows the subsystem 60 engaged with the distal sampling site 58. In this manner, the pressure monitoring function of the system is improved by removal of the dedicated reservoir 20 and stopcock 38 shown in FIGS. 1A-1C. That is, any reduction in signal fidelity caused by those dedicated components is removed. Also, elimination of an in-line reservoir dangling from the conduit line removes that potential cause of tangling with bedclothes or other such items. Furthermore, the fluid sampling aspect of the system is flexible such as that seen in FIG. 1A, with both proximal and distal sampling sites provided for use in different hospital settings, depending on need.

Exemplary details of the components of the system of FIGS. 2A-2B will be described below, however the system enables a method for sampling fluid from a medical pressure monitoring conduit line without a dedicated reservoir, though with the closed functionality of such a reservoir, and greatly improved convenience. In essence, the system enables as-needed fluid sampling from the pressure monitoring line by temporary attachment of a fluid reservoir to one of the sampling sites, occlusion of fluid communication between the reservoir and the conduit line proximal to the sampling site, withdrawal of a prime volume from the distal segment into the reservoir, taking of a blood sample, re-introduction of the prime volume into the conduit line, and removal of the fluid reservoir from the conduit line.

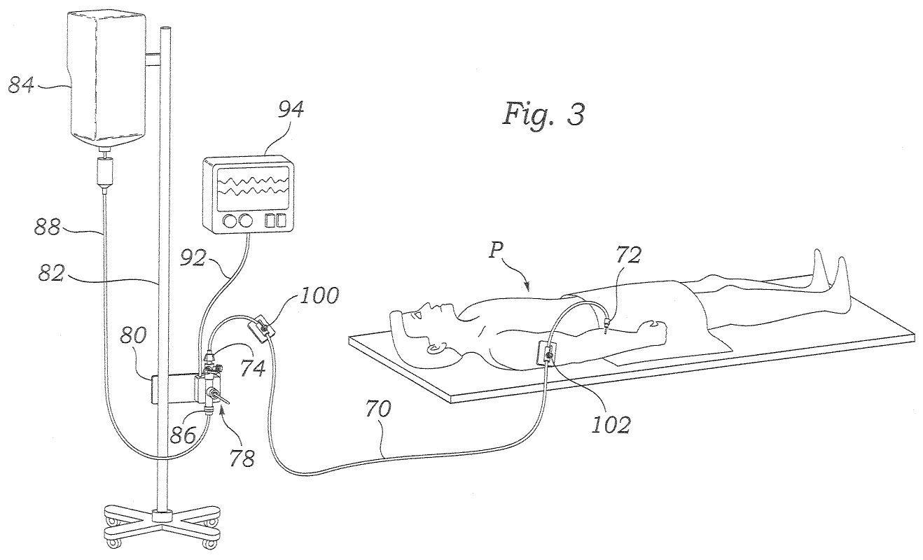

FIG. 3 illustrates an exemplary pressure monitoring system in a typical hospital room environment and connected to a patient P. The system shown in FIG. 3 is absent the detachable/disposable blood sampling subsystem 60 of FIG. 2, and therefore possesses a fairly low profile in the clinical setting which greatly enhances convenience when no blood sample is being taken. The pressure monitoring system comprises a conduit line 70 having a distal segment toward the patient and a proximal segment away from the patient. (As will be clear below, the terms proximal and distal segments will be used relative to either of two blood sampling sites.) The distal segment may terminate in a male luer connector 72 for attaching to a female luer connector (not shown) of an injection site, or other conduit leading to the patient.

As mentioned above, the exemplary blood sampling system advantageously works in conjunction with the pressure monitoring system. The proximal segment of the conduit line terminates in a female luer connector 74 attached to a stopcock (not shown) of a pressure transducer 78, such as a TruWave™ Disposable Pressure Transducer available from Edwards Lifesciences of Irvine, Calif. The pressure transducer 78 removably mounts to a bracket 80 which, in turn, may be secured to a conventional pole support 82 with the reservoir in a vertical orientation. A supply of flush solution 84 connects to a flush port 86 of the transducer 78 via tubing 88. Typically for adults, the flush solution 84 comprises a bag of physiological fluid such as saline surrounded by a pressurized sleeve that squeezes the fluid and forces it through the tubing 88. In addition, an infusion fluid supply (not shown) may be provided in communication with an infusion port of the stopcock 76. The pressure transducer 78 is thus placed in fluid communication with the arterial or venous system of the patient through the conduit line 70, and preferably includes a cable 92 and plug to connect to a suitable display monitor 94.

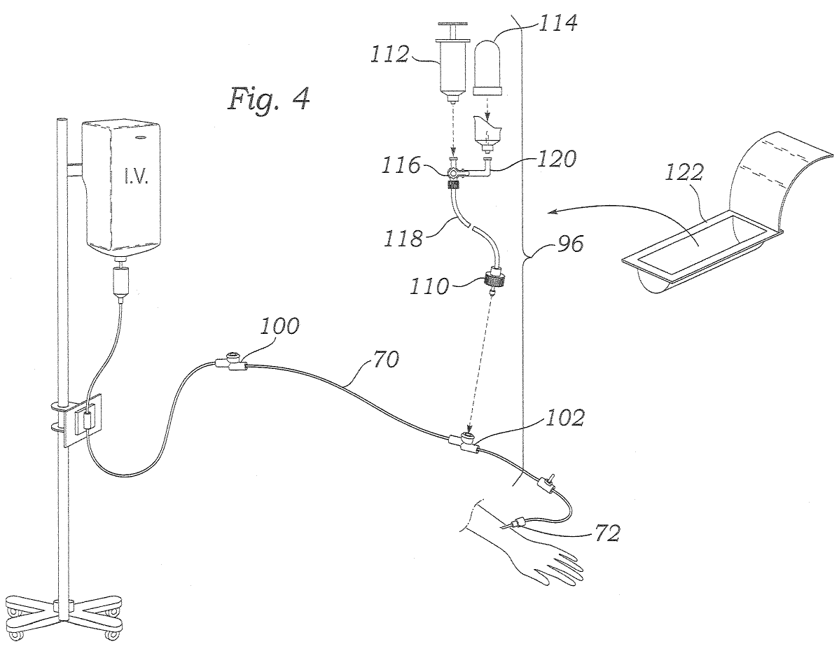

The blood sampling system 96 shown in FIG. 4 comprises at least one and desirably both proximal and distal fluid sampling sites 100, 102 in the conduit line (also seen in FIG. 3). Each sampling site 100, 102 defines a flow passage, preferably Z-shaped, adjacent a pre-slit septum (described below). With this configuration, a minimal amount of flush volume is needed to clear the line after sampling. The septum preferably comprises a split elastomeric disc which accepts a blunt cannula and reseals after each sample is drawn, reducing the potential for contamination and eliminating the danger of needle sticks. A similar sampling site is described in U.S. Pat. No. 5,135,489 to Jepson, et al.

FIG. 4 further illustrates an exploded packaged sterile kit that includes the aforementioned blood sampling subsystem 60 and integrates a blood sampling collection vessel with a pull-back reservoir, This is a single use, disposable accessory used to sample blood at either the proximal (OR) sampling site 100 or distal (ICU/CCU) sampling site 102. The kit desirably consists of the following elements:
Bypass cannula—110
Pull-back reservoir (shown as a simple syringe)—112
Sample collection vessel (Syringe or Vacutainer®)—114
3-position valve, or stop-cock—116
Short tube segment—118
Elbow—120
Sterile packaging—122

Because the sterile kit described above works in conjunction with one of the two sampling sites 100, 102, and samples blood that is present in the conduit line 70, all of these components together make up the fluid sampling system 96 of one embodiment of the present invention. However, it should be understood that some of the elements above, such as the elbow 120, are optional.

FIG. 4 best illustrates the disposable nature of most of the blood sampling system 96. Indeed, all of the disposable components of the system 96 are provided in the sterile package 122 for use at one of the two sample sites, and are then detached from the conduit line 70 and discarded. As mentioned, this greatly enhances the convenience of the system by eliminating the dedicated, in-line reservoir. However, as will be clear, the system remains closed during the sampling process and provides all of the safety benefits of a conventional dedicated reservoir. Moreover, as will be seen in greater detail below, the disposable components provided in the sterile package 122 are relatively simple, inexpensively produced items, which reduces the cost of the system even if multiple sterile packages 122 are used. Furthermore, because the reservoir 112 does not remain connected with conduit line 70, there is no need for a contamination sleeve or other special design to prevent blood stagnation.

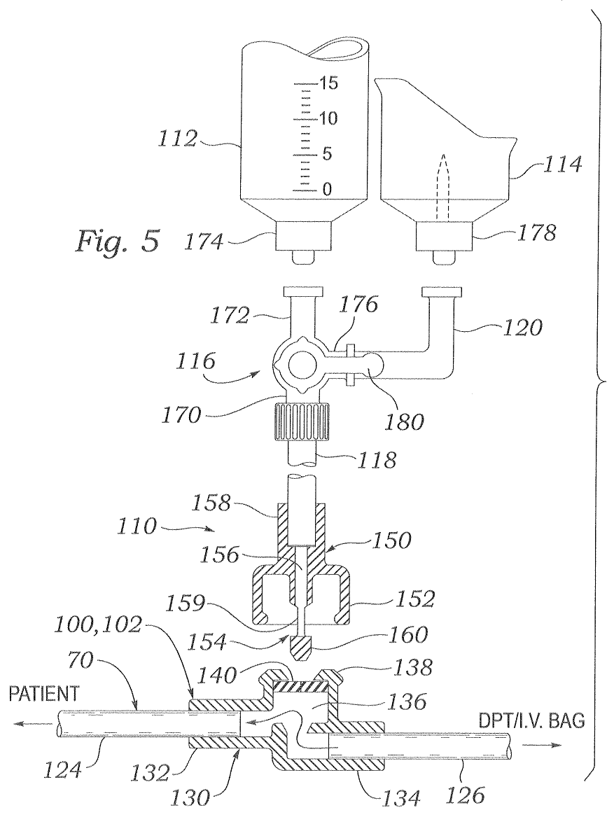

FIG. 5 is a close-up partial sectional view of an exemplary fluid sampling system 96 of one embodiment of the present invention. As mentioned, one of the two sampling sites 100, 102 within the conduit line 70 forms a part of the system 96. In the illustrated embodiment, the distal or patient segment 124 is to the left, while a proximal or pressure transducer/I.V. segment 126 is to the right The sampling site 100, 102 is shown as a conventional “Z-site,” so named because of the Z-shaped path taken by the fluid drip therethrough, as indicated. The sampling site 100, 102 includes a rigid housing 130 having a distal port 132 connected to the distal segment 124 and a proximal port 134 connected to the proximal segment 126, wherein fluid may flow freely through an inner chamber 136 in the housing between the proximal and distal ports. The sampling site 100, 102 further defines a sampling port 138 opening into the chamber 136 that is closed by an elastomeric septum or seal 140.

It should be emphasized at this point that although a particular blood sampling site (i.e., a Z-site configuration) is illustrated and described as a component of the blood sampling system, other in-line sampling sites could also be used. That is, the disposable blood sampling subsystem 60 described above may be designed to interact with other sampling sites than those illustrated. The disposable subsystem 60 is intended to be used with any flowthrough sampling site which includes a rigid housing having distal and proximal ports for connecting the sampling site within a fluid conduit line, and a sampling port to which the subsystem connects. The proximal, distal, and sampling ports all open to an internal chamber, and the sampling port is typically closed by an elastomeric seal or septum. As mentioned above, U.S. Pat. No. 5,135,489 to Jepson, et al. exemplifies this type of sampling site, though another similar sampling site which could be used is disclosed in U.S. Pat. No. 5,417,673 to Gordon.

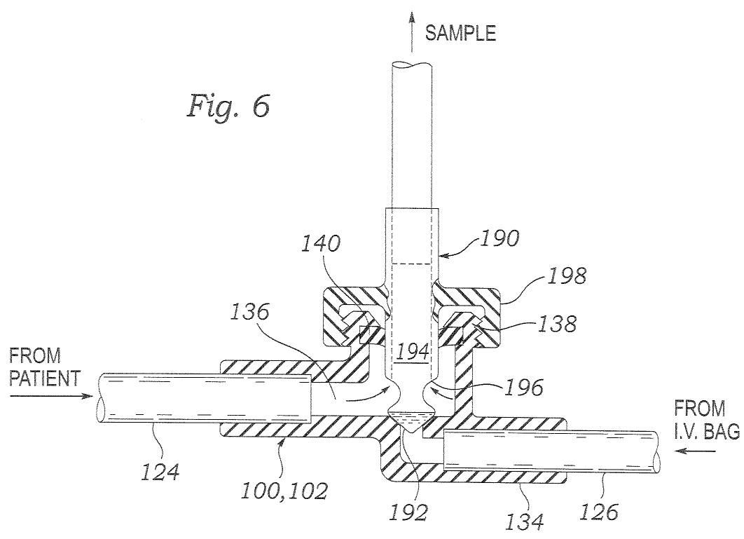

As seen in FIG. 5, the flowthrough bypass cannula 110 includes a generally tubular rigid body 150 defining a front fitting 152 shaped to engage the sampling port 138, and a bypass probe 154 that pierces the elastomeric seal 140, projects into the chamber 136 and occludes the proximal port 134 of the sampling site. The bypass cannula 110 further defines a central throughbore 156 in communication with a rear fitting 158 opposite the front fitting 152. The throughbore 156 extends part way along the bypass probe 154 and opens within the front fitting 152 at least one side passage 159. The terminal end of the probe 154 possesses an enlarged head or plug 160. The probe 154 may be configured in a number of ways, as will be seen, but includes a terminal end that mates with the opening to a passageway leading to one of the ports of the sampling site 100, 102, as seen in FIG. 6. The exemplary probe 154 further includes at least one opening such as the side passage 159 providing fluid communication between the throughbore 156 and the chamber 136 of the sampling site. The probe 154 therefore may be configured as a tubular member having a solid terminal end at plug 160, and diametrically opposed side passages 159 just before the plug.

The front fitting 152 is illustrated as a female cap member that fits over the male sampling port 138 of the sampling site 100, 102. In this regard, opposed flanges or ribs (not numbered) ensure an interference or snap fit and positive coupling of the bypass cannula 110 to the sampling site. Of course, other arrangements such as a threaded (e.g., luer) coupling may be provided. One desirable feature of the system is to maintain a good seal between the plug 160 and passage within the sampling site 100, 102. This can be accomplished between various materials (rigid or compressible) using mechanical pressure by way of a snap fit or a threaded engagement between the bypass cannula 110 to the sampling site 100, 102. Any such connection desirably provides constant mechanical force and accommodates tolerance variations typical of manufacturing processes.

The stopcock 116 connects to the rear fitting 158 of the bypass cannula 110. In this regard, the stopcock 116 may attach directly to the fitting 158, or via the intermediate tubing segment 118. A transparent tubing segment 118 is preferably utilized so that the clinician can monitor the formation of any bubbles within the aspirated fluid created by the suction of the reservoir 112. The short tubing segment 118 is included as a safety precaution against air bubble infusion. Because the syringe-type reservoir 112, bypass cannula 110, and tubing 118 are often not purged of air prior to sampling, an air pocket between the reservoir plunger and fluid may form during the draw back step. In the unlikely event of trapped air bubbles, they will be visible in the short (between 3 and 8 inches) tubing segment 118. In such a situation, the user would simply disconnect and dispose of the entire subsystem 60.

The stopcock 116 defines three ports: a first port 170 in communication with the rear fitting 158 of the bypass cannula, a second port 172 adapted to mate with a coupling 174 on the reservoir 112, and a third port 176 adapted to couple to one end of the elbow 120. The elbow 120 in turn includes a fitting on its opposite end that mates with a coupling 178 on the sample collection vessel 114. A handle 180 of the stopcock 116 may be manipulated into three positions to provide fluid communication between any two of the three ports, to the exclusion of the other. Again, the engagement of the various ports and connected elements may be through luer fittings, snap fittings, or the like. The use of standard luer connection fittings enables the users to substitute standardized components if they so choose

FIG. 6 illustrates a slightly modified flowthrough bypass cannula 190 engaged with a sampling site 100, 102. The bypass probe 194 is shown piercing the elastomeric seal 140 of the sampling site to enter the chamber 136. The tapered distal end 192 of the bypass probe 194 seats against a similarly tapered internal opening (not numbered) to the proximal port 134 of the sampling site. Because of one or more side openings 196 in the bypass probe 194, an inner throughbore (not shown) of the bypass cannula 190 opens to the chamber 136 within the sampling site. Inner threads on a front fitting 198 of the bypass cannula 190 mate with external threads formed on the sampling port 138 of the sampling site.

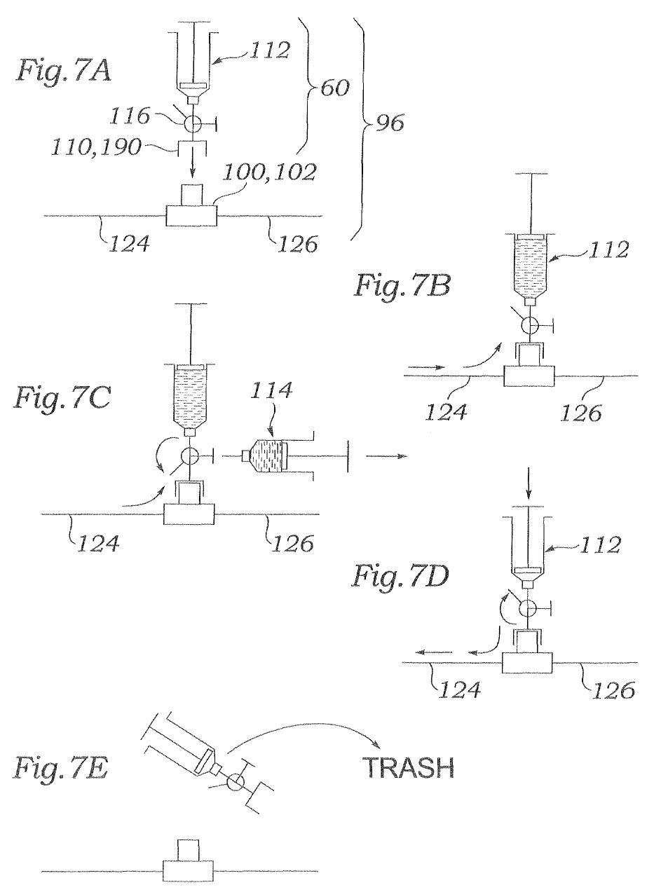

Operation of the fluid sampling system of one embodiment of the present invention will now be described with reference to the exemplary structures shown in FIGS. 5 and 6, and the schematic sequence of events shown in FIGS. 7A-7E.

First, the fluid sampling subsystem 60 is removed from the sterile package 122 (FIG. 4) and the appropriate components are connected with the bypass cannula 110, 190 engaging the sampling port 138 of the sampling site 100, 102 (FIG. 7A). As seen in FIG. 6, this causes the bypass probe 154, 194 to pass through the elastomeric septum 140 into the chamber 136. The length of the probe 194 and its configuration is such that its distal tip 192 engages and occludes the opening to the proximal port 134 of the sampling site. The single step of engaging the cannula 110 to the sampling site 100, 102 places the reservoir 112 into fluid communication with the distal segment 124 of the conduit line and simultaneously blocks flow between the proximal segment 126 and the sampling site chamber 136.

The step of engaging the bypass cannula 110, 190 with the sampling site 100, 102 is desirably accomplished with the handle 180 of the stopcock 116 in a position that blocks fluid flow through the stopcock; that is, the first port 170 is closed. After secure engagement of the bypass cannula 110, 190 with the sampling site, the clinician manipulates the stopcock handle 180 into a position that permits fluid flow from the sampling site through the bypass cannula 110, 190 and to the reservoir 112; that is, the first port 170 communicates with the second port 172.

Retraction of a plunger of the reservoir 112 creates a negative pressure differential such that a fluid sample from the distal segment 124 is drawn into the chamber 136 and into the reservoir 112 (FIG. 7B). The reservoir 112 has a sufficient volume, typically 5-15 ml, to draw blood from the patient P past both sampling sites 100, 102. If blood is being taken from distal sampling site 100 in the ICU/CCU, the prime volume is typically about 5 ml, whereas if blood is taken from the proximal sampling site 102 in the OR, the prime volume is typically about 12 ml. Please note that the syringe-type reservoir 112 could be used, or another manual draw type reservoir, or even mechanically-assisted fluid draw device.

Once all of the saline and mixed saline and blood has been pulled into the reservoir 112, the clinician can then take a sample of undiluted blood from the site 100, 102. To do so, he/she manipulates the stopcock handle 180 into a position that blocks flow to the reservoir 112 and permits flow between the bypass cannula 110 and the third port 176 leading to the elbow 120 and sample collection vessel 114. As indicated in FIG. 7C, a sample of blood is then drawn into the collection vessel 114 through action of a plunger, or as soon as the stopcock handle 180 opens the flow, if the collection vessel is an evacuated container (e.g., Vacutainer®). The sample collection vessel 114 is designed to be detached from the elbow 120 or directly from the stopcock 116 for remote analysis of the blood therein. It should be understood that in one use of the disposable blood sampling subsystem 60 more than one sample may be taken by simply engaging more than one collection vessel 114 in sequence.

After the desired sample is taken, the stopcock handle 180 is again manipulated into a position that closes off the third port 176 and opens flow between the first and second ports 170, 172 (i.e., between the sampling site 100, 102 and reservoir 112). Subsequently, the blood and other fluids drawn into the reservoir 112 during the sample priming operation are re-infused by depressing the reservoir plunger (FIG. 7D) into the distal segment 124, and ultimately back to the patient. It should be noted that during this re-infusion step the bypass probe 154, 194 remains occluding the proximal port 134 of the sampling site so that fluid from the reservoir 112 does not travel into the proximal segment 126 toward the pressure transducer.

It is important to understand that the sampling system 96 provides a closed reservoir 112 since the priming volume that ensures a pure sample of blood reaches the sampling site 100, 102 remains within the system 96 and is re-infused into the patient without removal of the reservoir. That is, because the reservoir 112 as a part of the sampling subsystem 60 remains connected to the sampling site 100, 102 during the entire process until re-infusion, the conduit line is not “broken,” meaning the reservoir is not detached between drawing the prime volume and re-infusing it. Of course, as mentioned above, if the clinician notices bubbles have formed within the reservoir 112 or adjacent tubing, the subsystem 60 can be removed from the sampling site 100, 102 without re-infusing the prime volume, which is then also discarded. This is a significant advantage over earlier “dedicated” reservoirs which could not be removed and had to be operated with great care to prevent formation of bubbles.

Finally, as indicated in FIG. 7E, after obtaining a blood sample the clinician detaches the fluid sampling subsystem 60 from the sampling site 100, 102 and discards it. Again, this is preferred over dedicated reservoirs which can get tangled with sheets and such.

The reader will now appreciate certain benefits of the improved fluid sampling system as follows:
The pull back reservoir 112 desirably comprises a simple inexpensive syringe. Because this is a single use device no contamination shield is required.
Elimination of the pull back reservoir from the pressure monitoring system eliminates the chances of air bubbles, residual blood, and makes priming and initial set-up much easier.
By eliminating the reservoir from the pressure monitoring system there is no impact to the pressure wave form fidelity.
The pull back reservoir is no longer integrated into the tubing system. This eliminates the tangling or snagging with the patients bedding or other equipment. Elimination of the reservoir makes for a much cleaner, or less cluttered, system.
Blood pressure monitoring kits can be configured with two Z-sites so that both ICU and OR clinicians can have easy access to blood samples. A single “universal” blood pressure monitoring kit can serve both the ICU and the OR user.
Products that are currently available require the user to first manipulate the reservoir, and valve. They then have to reposition their hands to take a sample from the Z-site. Then they have to reposition their hands back to the reservoir and valve. This sequence requires the user to reposition three times. The advantage of the present system is that all sampling activities happen with one single user positioning.
Because the reservoir is only used once there is no need for a complex design, nor is there a need for a contamination shield. A standard syringe can be incorporated into the kit configuration thereby providing a low cost sampling system.

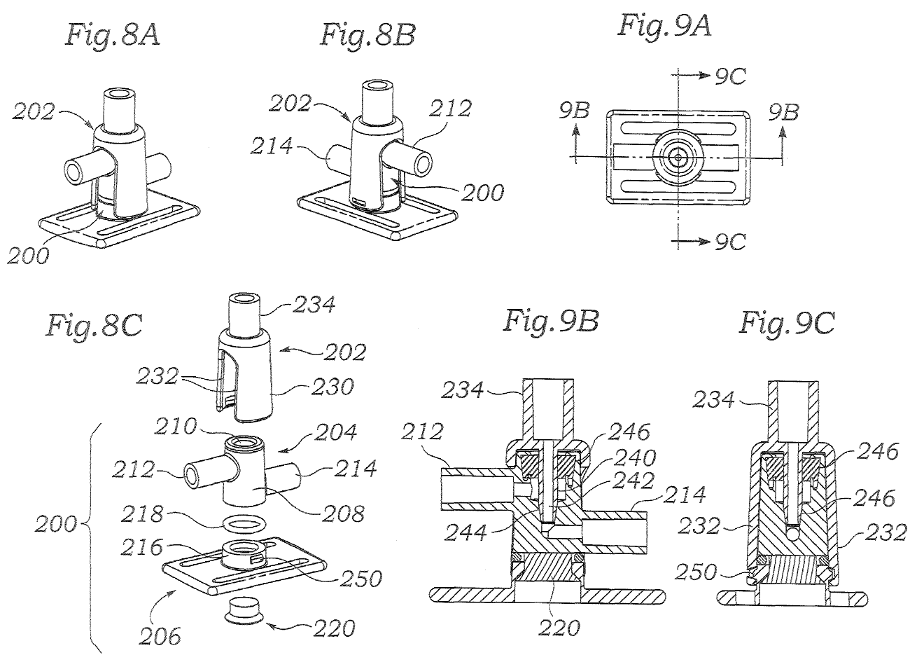

The bypass function (i.e., excluding the proximal segment) of the sampling system of the present invention can be achieved in a number of ways. FIGS. 8A-8C and 9A-9E illustrate in great detail an alternative exemplary sampling site 200 and bypass cannula 202. The sampling site 200 is shown exploded in FIG. 8C and features a main body 204 adapted to project upward from a base plate 206. The base plate 206 includes slots for securing it with tape, for example, to a base surface. The main body 204 is desirably rigid molded plastic having a central column 208 with a closed bottom and a sampling port 210 opening upward. A proximal port 212 projects horizontally from one side of the column 208, while a distal port 214 projects horizontally in the opposite direction but offset vertically. The main body 204 mounts on top of a tubular flange 216 on the base plate 206 via an intermediate O-ring 218. As seen in the sectional views of FIGS. 9B and 9C, a plug member 220 extends upward into a countersunk bore in the base plate 206 to contact the underside of the main body 204. Desirably, the main body 204 and the plug member 220 are attached via adhesive, or other similar expedient.

The bypass cannula 202 is similar to those described above and includes a front fitting 230 bifurcated into a pair of fingers or skirts 232. The front fitting 230 positively engages the sampling port 210 of the sampling site 200, while a rear fitting 234 projects upward. The vertical gaps between the bifurcated skirts 232 receive the outwardly projecting proximal and distal ports 212, 214.

With reference to FIGS. 9B and 9C, the bypass cannula 202 further includes a bypass probe 240 aligned along a central axis defining a throughbore 242 therein and terminating in an open and tapered distal tip 244. When the bypass cannula 202 couples to the sampling site 200, the bypass probe 240 projects through an elastomeric septum 246 provided at the opening of the sampling port 210. The length of the bypass probe 240 is sufficient so that the tapered tip 244 extends into a central chamber of the sampling site main body 204 and into engagement with a similarly tapered opening (not numbered) that communicates with a passageway opening leading to the distal port 214. Upon engagement between the bypass cannula 202 and sampling site 200, the throughbore in the bypass probe 240 and rear fitting 234 is placed in fluid communication with the passageway through the distal port 214, to the exclusion of the proximal port 212. That is, instead of forming the bypass probe 240 with a solid distal end and side openings as before, it is a continuous tubular member open only at its distal end, so that engagement of the passageway opening with the distal port 214 excludes communication between the proximal port 212 and the throughbore 242.

The bifurcated skirts 232 of the bypass cannula 202 extend axially farther than the length of the bypass probe 240. The bypass cannula 202 therefore includes the safety feature of a blunt-tipped probe 240 which is also protected from undesirable contact or damage by the generally parallel and laterally adjacent skirts 232, The skirts 232 each have on their lowermost ends small internal depressions that receive similarly-shaped teeth 250 on the tubular flange 216 of the sampling site 200. Furthermore, the external shape of the central column 208 of the sampling site main body 204 is slightly conical, though the skirts 232 are not and they are slightly outwardly biased upon engagement with the main body 204. Engaging the bypass cannula 202 onto the sampling site 200 involves pressing the cannula downward such that the probe 240 extends through the elastomeric septum 246 and the bifurcated skirts 232 flex outward until the lower depressions are engaged with the teeth 250. Desirably, the engagement provides an audible “snap,” and the coupling is made more secure by the frictional contact of the skirts 232 against the intermediate O-ring 218. Once the bypass cannula 202 and sampling site 200 are fully engaged, the tapered tip 244 of the bypass probe 240 accurately seats within the tapered opening in the sampling site main body 204. The procedure for taking a fluid sample using the combined bypass cannula 202 and sampling site 200 is similar to that described above.

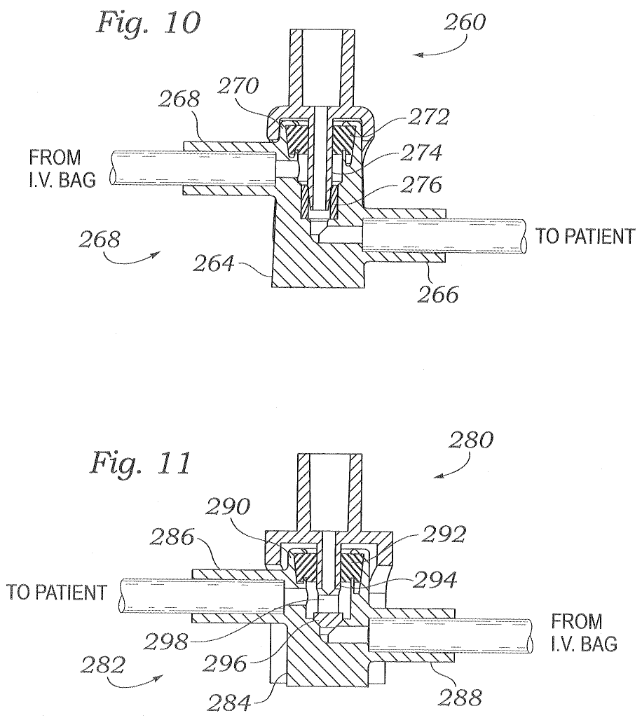

FIG. 10 illustrates an alternative bypass cannula 260 and sampling site 262 combination of one embodiment of the present invention which is very similar to that shown in FIGS. 8-9. As before, the sampling site 262 includes a main body 264 defining an internal chamber opening at a distal port 266, a proximal port 268, and a sampling port 270. An elastomeric seal or septum 272 occludes the sampling port 270 until the bypass cannula 260 engages the sampling site 262 and a bypass probe 274 projects therethrough. As above, the bypass probe 274 has a tapered tip, but instead of fitting into a tapered seat within the main body 264, it expands within and contacts the bore of a tubular sealing member 276. The sealing member 276 is made of a compressible material such as silicone rubber which provides good sealing contact against the bypass probe 274. It will thus be apparent that engagement of the bypass cannula 260 over the sampling site 262 enables a fluid flow between the bypass cannula throughbore and the distal port 266, thus bypassing the proximal port 268.

FIG. 11 illustrates another alternative bypass cannula 280 and sampling site 282 combination of one embodiment of the present invention which is similar to that shown in FIG. 5. As before, the sampling site 282 includes a main body 284 defining an internal chamber opening at a distal port 286, a proximal port 288, and a sampling port 290. An elastomeric seal or septum 292 occludes the sampling port 290 until the bypass cannula 280 engages the sampling site 282 and a bypass probe 294 projects therethrough. As with the embodiment of FIG. 5, the bypass probe 294 terminates in an enlarged and tapered tip 296. The tip 296 acts as a plug to contact and occlude a passageway leading to the proximal port 288. One or more side openings 298 provided in the bypass probe 294 enable fluid communication between the internal chamber of the main body 284 and the throughbore of the bypass cannula 280. It will thus be apparent that engagement of the bypass cannula 280 over the sampling site 282 enables a fluid flow between the bypass cannula throughbore and the distal port 286, to the exclusion of the proximal port 288. To further enhance the ability of the bypass probe 294 to plug the opening to the proximal port 288, the tapered tip 296 may be supplemented with an elastomeric head or other compressible coating.

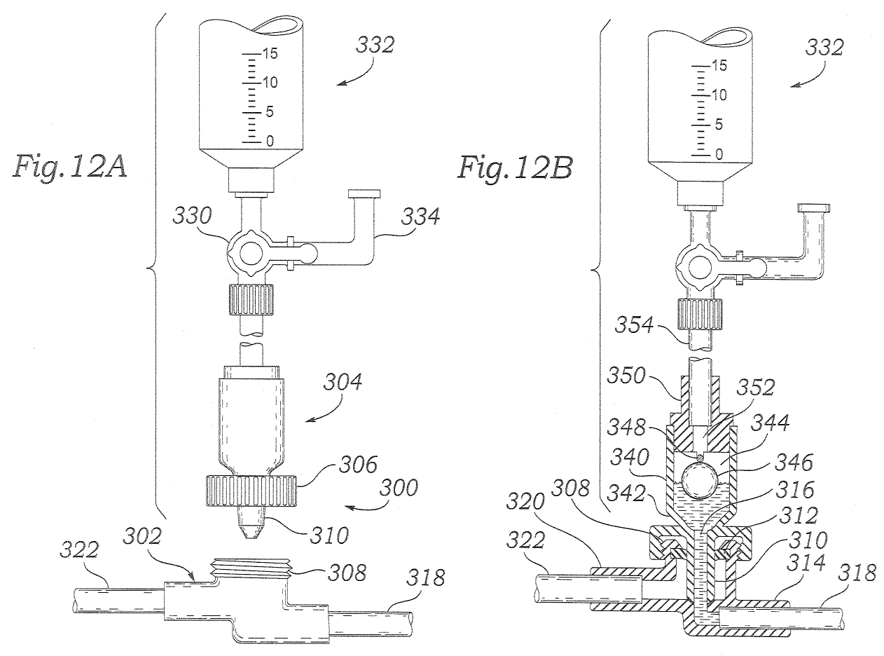

FIGS. 12A and 12B illustrate a still further alternative bypass cannula 300 and sampling site 302 combination, as well as an exemplary bubble trap 304 that may be used therewith. The bypass cannula 300 has a threaded front fitting 306 adapted to mate with a threaded sampling port 308 of the sampling site 302. As before, the bypass cannula 300 includes a bypass probe 310 sized to extend through an elastomeric seal or septum 312 into engagement with a passage leading to a distal port 314 of the sampling site 302. The bypass probe 310 is similar to the bypass probe 240 of FIGS. 8 and 9 in that it is a continuous tube having a central throughbore 316. The engagement of bypass cannula 300 with the sampling site 302 therefore connects the throughbore of the cannula with the distal port 314 and a distal segment 318 of a conduit line leading to a patient, and excludes a proximal port 320 and a proximal segment 322 from the flow path.

The bypass cannula 300 connects through the bubble trap 304 to a 3-way stopcock 330, and from there to a reservoir 332 and elbow conduit 334. Although not shown, a sampling collection vessel may be connected to the opposite end of the elbow conduit 334. Indeed, the system functions in a similar manner to those described above, with the exception of the interposition of the bubble trap 304.

With reference to FIG. 12B, the bubble trap 304 includes a generally tubular outer body 340 having a conical lower end 342 and defining a float chamber 344 therewith. The lower end 342 opens into the central throughbore 316 of the bypass probe 310. A ball valve 346 floats on any fluid within the chamber 344, and nests within a similarly shaped inner surface at the bottom of the float chamber 344. A flow separator 348 projects downward from an upper cap 350 of the bubble trap 304. Passages around the flow separator 348 leading to an upper throughbore 352 within the cap 350. The upper cap 350 receives the optional flexible tubing 354 leading to the 3-way stopcock 330.

In use, fluid drawn into the reservoir 332 passes upward through the bubble trap 304 without hindrance. Bubbles that may form from the suction created by the reservoir 332 rise to the fluid surface layer within the chamber 344 and pop. Upon re-infusion from the reservoir 332, the fluid passes downward through the bubble trap 304, bypass cannula 300, and into the distal port 314 and distal segment 318 of the conduit line. Once the fluid level within the chamber 344 lowers enough, the ball valve 346 contacts the shaped inner surface of the float chamber 344 to prevent any air or bubbles being passed into the conduit line.

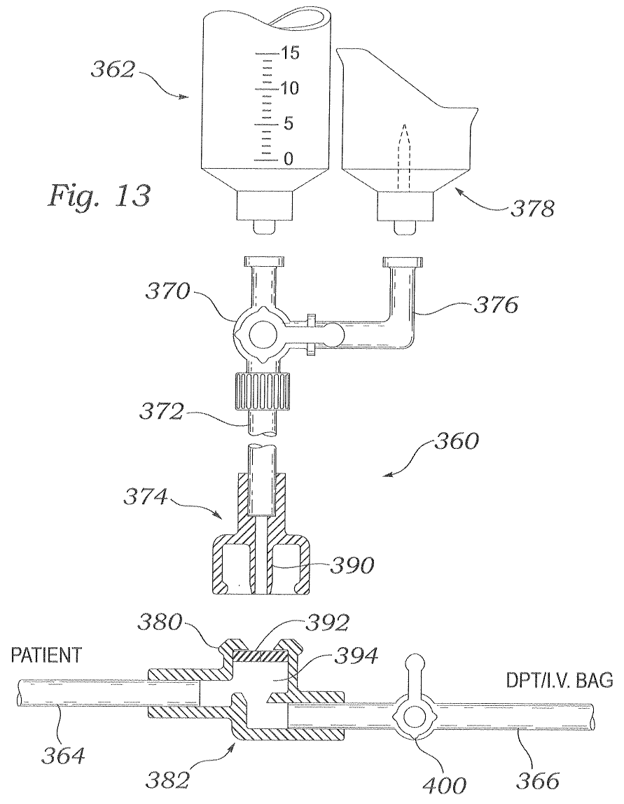

FIG. 13 is an alternative disposable fluid sampling system 360 of one embodiment of the present invention that again permits fluid to be drawn into and expelled from a removable reservoir 362 to and from a distal segment 364 of a conduit line while occluding a proximal segment 366. Although not shown, the distal and proximal segments 364, 366 desirably formed a part of a pressure monitoring conduit line where the distal segment 364 communicates with a fluid system of the patient and proximal segment 366 connects to a disposable pressure transducer (DPT) and an intravenous (I.V.) drip. The fluid sampling system 360 resembles somewhat that shown in FIG. 5, wherein a disposable subsystem comprises the reservoir 362, a 3-way stopcock 370, an optional length of flexible tubing 372, a flowthrough bypass cannula 374, an optional elbow conduit 376, and a sample collection vessel 378. The bypass cannula 374 engages a sampling port 380 of the sampling site 382 positioned in the conduit line between the distal segment 364 and proximal segment 366.

In contrast to the earlier-described embodiments, a hollow probe 390 on the bypass cannula 374 has a length sufficient to extend through a slit septum 392 within the sampling port 382 into an inner chamber 394 of the sampling site 382, but not long enough to engage any passageway openings therein. Instead, the probe 390 extends just passed the septum 392 into the inner chamber 394 and remains in fluid communication with both the distal segment 364 and proximal segment 366. In other words, the probe 390 does not in itself provide the bypass function described above.

The fluid sampling system 360 further includes a 2-way stopcock 400 positioned adjacent to the sampling site 382 in the proximal segment 366 of the conduit line. Alternatively, the 2-way stopcock 400 could be incorporated into the sampling site 382, although such a design is relatively more expensive to manufacture. The stopcock 400 provides the bypass function of the fluid sampling system.

In use, the clinician manipulates the stopcock 400 to disconnect the proximal segment 366 of the conduit line from the sampling site 32. The disposable subsystem is removed from its sterile package (e.g., the sterile package 122 of FIG. 4) and the appropriate components are connected with the bypass cannula 374 engaging the sampling port 380 of the sampling site 382. The probe 390 passes through the elastomeric septum 392 into the chamber 394.

The step of engaging the bypass cannula 374 with the sampling site 382 occurs while the stopcock 370 blocks fluid flow therethrough. After secure engagement of the bypass cannula 374 with the sampling site 382, the clinician manipulates the stopcock 370 into a position that permits fluid flow from the distal segment 364 and sampling site 382 through the bypass cannula 374 and to the reservoir 362. Retraction of a plunger (not shown) of the reservoir 362 creates a negative pressure differential such that a fluid sample from the distal segment 364 is drawn into the chamber 394 and into the reservoir 362.

Once all of the saline and mixed saline and blood has been pulled into the reservoir 362, the clinician can then take a sample of undiluted blood from the site 382. To do so, he/she manipulates the stopcock 370 into a position that blocks flow to the reservoir 362 and permits flow between the bypass cannula 374 and the elbow conduit 376 and sample collection vessel 378.

After the desired sample is taken, the clinician again manipulates the stopcock 370 into a position that closes off the elbow conduit 376 and opens flow between the reservoir 362 and sampling site 382. Blood and other fluids drawn into the reservoir 362 during the sample priming operation are re-infused by depressing the reservoir plunger into the distal segment 364, and ultimately back to the patient. It should be noted that during this re-infusion step the stopcock 400 remains closed so that fluid re-infused from the reservoir 362 does not travel into the proximal segment 366 toward the DPT. After re-infusion of the prime volume the clinician manipulates the stopcock 370 to close off the communication between the sampling port 380 and reservoir 362 and removes and discards the disposable subsystem by disconnecting the bypass cannula 374 from the sampling site 382. The clinician opens the stopcock 400 and the “normal” operation of the conduit line resumes, such as a flushing drip and pressure monitoring.

Again, the sampling system 360 provides a closed reservoir 362 since the printing volume that ensures a pure sample of blood reaches the sampling site 382 remains within the system and is re-infused into the patient without removal of the reservoir. That is, because the reservoir 362 as a part of the disposable subsystem remains connected to the sampling site 382 during the entire process until re-infusion, the conduit line is not “broken,” meaning the reservoir is not detached between drawing the prime volume and re-infusing it. Of course, as mentioned above, if the clinician notices bubbles have formed within the reservoir 362 or adjacent tubing, the disposable subsystem can be removed from the sampling site 382 without re-infusing the prime volume, which is then also discarded.

The system 360 of FIG. 13 functions in a similar manner as the earlier-described embodiments, in that the sampling subsystem including the fluid draw reservoir is removable and disposable. Moreover, the reservoir is closed because it remains in place during the fluid draw and re-infusion. However, if bubbles are detected in the reservoir or intermediate tubing the fluid draw can be discarded which is not possible with a dedicated reservoir. All of the systems of the present invention provide a removable/disposable closed reservoir that can be isolated from the proximal or upstream segment of the conduit line.

In contrast to the earlier-described embodiments of the present invention, the bypass feature of the fluid sampling system 360 is enabled by the proximal stopcock 400 rather than by simple engagement of a bypass cannula with the sampling site. One advantage with such a system is the elimination of the need for special components such as the bypass probes described above, and therefore there is an attendant reduction in manufacturing costs. A potential drawback, however, is the possibility of including some saline in the blood sample taken because of the spacing between the stopcock 400 and the sampling site 382. That is, the reservoir 362 draws most of the saline and mixed saline and blood therein, though some saline or diluted blood may remain in the passages that are not in the direct line of reservoir flow. Also, because a second stopcock 400 is included, the system 360 is less elegant and not as simple to use.

While the invention has been described in its preferred embodiments, it is to be understood that the words which have been used are words of description and not of limitation. Therefore, changes may be made within the appended claims without departing from the true scope of the invention.

What is claimed is:

1. A medical system for sampling of a fluid system of a patient for use with a conduit line with a proximal segment adapted to be supplied with a physiological fluid and a distal segment adapted to be in communication with the fluid system of a patient, and a fluid sampling site in the conduit line, the sampling site including a housing having a distal port connected to the distal segment and a proximal port connected to the proximal segment, wherein fluid may flow freely through a chamber in the housing between the proximal and distal ports, the sampling site further defining a sampling port open to the chamber and closed from the exterior by an elastomeric seal, the medical system comprising:
a flowthrough bypass cannula having a body shaped to engage the sampling port and a bypass probe having a throughbore and being sized to pierce the elastomeric seal, project into the chamber and place the throughbore into fluid communication with the distal port of the sampling site while occluding the proximal port from the throughbore, wherein a fluid sample may be drawn into the bypass cannula from the distal segment of the conduit line to the exclusion of the proximal segment.

2. The system of claim 1, wherein the bypass probe terminates in a tip that contacts a passageway opening in the fluid sampling site housing leading to one of the distal or proximal ports when the bypass cannula engages the sampling site, and the tip is tapered and matches the passageway opening.

3. The system of claim 1, wherein the bypass probe terminates in a closed tip that contacts and occludes a passageway opening in the fluid sampling site housing leading to the proximal port when the bypass cannula engages the sampling site, and the bypass probe includes at least one side opening open to the chamber in the sampling site housing.

4. The system of claim 3, wherein the bypass probe tip is at least partly compressible.

5. The system of claim 1, wherein the bypass probe defines a continuous tubular member and terminates in an open tip that contacts a passageway opening in the fluid sampling site housing leading to the distal port when the bypass cannula engages the sampling site.

6. The system of claim 5, further including a sealing member positioned in the passageway opening that contacts and seals against the open tip of the bypass probe.

7. The system of claim 1, wherein the bypass cannula and sampling site include mating structure such that they engage with a positive snap fit.

8. A system for sampling fluid from a medical conduit line including a disposable subsystem, comprising:
a conduit line with a proximal segment adapted to be supplied with a physiological fluid and a distal segment adapted to be in communication with a fluid system of a patient;
a fluid sampling site in the conduit line having an internal chamber in communication with all of: a proximal port connected to the proximal segment, a distal port connected to the distal segment, and a sampling port closed by an elastomeric seal;
a disposable sampling subsystem for engaging the fluid sampling site including:
a bypass cannula adapted to engage the sampling port of the sampling site and simultaneously occlude the proximal port therein;
a stopcock having three ports, a first port in communication with the bypass cannula;
a fluid reservoir connected to a second port of the stopcock;
a fluid sampling vessel in fluid communication with the third port of the stopcock;
wherein in a first position the stopcock enables fluid communication between the first and second ports so that fluid may be drawn into the reservoir from the distal segment, and in a second position the stopcock enables fluid communication between the first and third ports so that fluid may be drawn into the fluid sampling vessel from the distal segment.

9. The system of claim 8, further including:
a pressure transducer connected to the proximal segment of the conduit line for sensing the pressure of the fluid system of a patient through the fluid in the conduit line.

10. The system of claim 8, wherein the bypass cannula includes a bypass probe terminating in a closed tip that contacts and occludes a passageway opening in the fluid sampling site housing leading to the proximal port when the bypass cannula engages the sampling site, and the bypass probe includes at least one side opening open to the chamber in the sampling site housing.

11. The system of claim 8, wherein the bypass cannula includes a continuous tubular bypass probe terminating in an open tip that contacts a passageway opening in the fluid sampling site housing leading to the distal port when the bypass cannula engages the sampling site.

12. The system of claim 8, wherein the system further includes a stopcock positioned adjacent the proximal port of the sampling site for occluding the proximal port when fluid is drawn into and expelled from the reservoir and drawn into the fluid sampling container,.

13. The system of claim 8, further including:
a bubble trap connected to the bypass cannula between the sampling site and reservoir that prevents bubbles from entering the sampling site from the bypass cannula.

14. A method for sampling blood from a medical fluid conduit line using a disposable subsystem, the conduit line having a proximal segment supplied with a physiological fluid and a distal segment in communication with the blood system of a patient, the conduit line also having a fluid sampling site defining an internal chamber in communication with all of: a proximal port connected to the proximal segment, a distal port connected to the distal segment, and a sampling port closed by an elastomeric seal, the method comprising:
providing a disposable sampling subsystem for engaging the fluid sampling site including a flowthrough bypass cannula having a probe, a fluid reservoir, and a fluid sampling container;
engaging the bypass cannula with the sampling site so that the probe passes through the elastomeric seal;
opening fluid communication between the bypass cannula and reservoir while closing fluid communication between the bypass cannula and fluid sampling container;
closing fluid communication between the bypass cannula and proximal segment of the conduit line and drawing fluid into the reservoir from the distal segment through the bypass cannula until blood enters the sampling site;
opening fluid communication between the bypass cannula and fluid sampling vessel while closing fluid communication between the bypass cannula and reservoir;
drawing blood into the fluid sampling vessel from the distal segment;
maintaining the bypass cannula engaged with the sampling site until a sample of blood is drawn into the fluid sampling container; and
detaching the bypass cannula from the sampling site and disposing of the sampling subsystem.

15. The method of claim 14, wherein the disposable sampling subsystem further includes a stopcock having three ports, a first port in communication with the bypass cannula, a second ports in communication with the fluid reservoir, and a third port in communication with the fluid sampling container, wherein following steps of the method are enabled by manipulating the stopcock:
opening fluid communication between the bypass cannula and reservoir while closing fluid communication between the bypass cannula and fluid sampling container; and
opening fluid communication between the bypass cannula and fluid sampling vessel while closing fluid communication between the bypass cannula and reservoir.

16. The method of claim 14, further including:
providing a pressure transducer connected to the proximal segment of the conduit line and sensing the pressure of the fluid system of a patient through the fluid in the conduit line.

17. The method of claim 14, wherein the step of opening fluid communication between the bypass cannula and reservoir while closing fluid communication between the bypass cannula and fluid sampling vessel is accomplished by the step of engaging the bypass cannula with the sampling site.

18. The method of claim 17, wherein the probe of the bypass cannula has a size sufficient to contact and occlude a passageway opening leading to the proximal port of the sampling site.

19. The method of claim 17, wherein the probe of the bypass cannula has a size sufficient to contact a passageway opening leading to the distal port of the sampling site and exclude fluid communication between the proximal port and the distal port.

20. The method of claim 14, wherein the method further includes providing a stopcock adjacent the proximal port of the sampling site for occluding the proximal port when fluid is drawn into and expelled from the reservoir and drawn into the fluid sampling container.
